(19) United States
(12) Patent Application Publication
(10) Pub. No.: US 2020/0381079 A1
(43) Pub. Date: Dec. 3, 2020

(54) METHODS FOR DETERMINING SUB-GENIC COPY NUMBERS OF A TARGET GENE WITH CLOSE HOMOLOGS USING BEADARRAY

(71) Applicant: Illumina, Inc., San Diego, CA (US)

(72) Inventor: Yong Li, San Diego, CA (US)

(21) Appl. No.: 16/890,982

(22) Filed: Jun. 2, 2020

Related U.S. Application Data
(60) Provisional application No. 62/856,281, filed on Jun. 3, 2019.

(51) Int. Cl. G16B 20/10

(52) U.S. Cl. CPC G16B 20/10

(57) ABSTRACT

Presented herein are methods and compositions for copy number estimation of a target gene with close homologs, comprising determining sub-genic copy numbers. The methods are useful for estimating copy numbers of clinically important genes with high sequence similarity between gene of interest and their homologs, including non-functional pseudogenes.

### CROSS-REFERENCE TO RELATED APPLICATION

The present application claims the benefit of U.S. Provisional Application No. 62/856,281, which was filed on Jun. 3, 2019 and is entitled “METHODS FOR DETERMINING SUB-GENIC COPY NUMBERS OF A TARGET GENE WITH CLOSE HOMOLOGS USING BEADARRAY,” and which is incorporated herein by reference in its entirety.

### BACKGROUND

Genotyping is challenging. For example, spinal muscular atrophy is caused by loss of the functional survival of motor neuron 1 (SMN1) gene but retention of the paralogous SMN2 gene. Due to the near identical sequences of SMN1 and its paralog SMN2, analysis of this region has been challenging. As another example, CYP2D6 is involved in the metabolism of 25% of all drugs. Genotyping CYP2D6 is challenging due to its high polymorphism, the presence of common structural variants (SVs), and high sequence similarity with the gene's pseudogene paralog CYP2D7.

The sequences together with the copy numbers of human genes determine their functions in disease and drug responses. However, for many clinically important genes, copy number estimation can be challenging due to high sequence similarity between gene of interest and their homologs, including non-functional pseudogenes. As such, there remains a great need for improved copy number estimation methodologies.

### BRIEF SUMMARY

Presented herein are methods and compositions for determining sub-genic copy numbers of a target gene with close homologs. In some exemplary embodiments, the methods use data from an array. In some aspects, the array is a genotyping array, such as, for example, a bead array.

Also presented herein is a method for genotyping cytochrome P450 Family 2 Subfamily D Member 6 (CYP2D6) gene, the method comprising, under control of a hardware processor: receiving quantitative data comprising nucleotide sequence information at one or more specific sites of cytochrome P450 Family 2 Subfamily D Member 6 (CYP2D6) gene or cytochrome P450 Family 2 Subfamily D Member 7 (CYP2D7) gene, said quantitative data obtained from a sample of a subject analyzed; determining a first number of informative signals from each of said one or more specific sites; determining a first normalized number of informative signals from each of said one or more specific sites; determining an aggregated informative signal for each of a plurality of target regions, and determining a total copy number of one or more CYP2D6 genes, sub-genic regions or pseudogenes using a Gaussian mixture model.

In certain aspects, determining (i) a first normalized number of informative signals comprises normalizing based on the length of a gene or sub-genic region. In certain aspects, determining (i) a first normalized number of informative signals comprises normalizing based on genomic GC content of a gene or sub-genic region.

In certain aspects, the extracted informative signals are aggregated through an arithmetic mean. In certain aspects, the arithmetic mean comprises: Ts=Σsl Rsl/L, or Ts=Σsl exp(rsl)/L, where r and R are the log scale or linear scaled normalized signal respectively, s and l indicate the sample and (informative) loci, and L is the total number of loci.

In certain aspects, the extracted informative signals are aggregated through a geometric mean. In certain aspects, the geometric mean comprises: Tₛ=Σₛₗ Rₛₗ/L, or Tₛ=Σₛₗ exp(rₛₗ)/L.

In certain aspects, a weighted version of the signal aggregation method is applied. In certain aspects, the weighted version of the signal aggregation method comprises: Ts=exp(Σsl log(Rsl)/L), where σl2 is the variance of signal of a given loci across all samples.

In certain aspects, the method further comprises, following signal aggregation, a centering step to remove batch effect common to all samples.

In certain aspects, the Gaussian mixture model comprises a restricted expectation maximization (EM) algorithm. In certain aspects, the restricted EM algorithm estimates the means and variances of intensity signals associated with difference copy number states. In certain aspects, the restricted EM algorithm estimates the priors associated to the copy number states. In certain aspects, the Gaussian mixture model comprises a plurality of Gaussians each representing a different integer copy number, given the first normalized number of the quantitative sequence information from the one or more specific sites of the CYP2D6 gene. In certain aspects, determining a total copy number of one or more CYP2D6 genes, sub-genic regions or pseudogenes comprises, for one of a plurality of CYP2D6 gene-specific bases, determining a most likely combination, of a plurality of possible combinations each comprising a possible copy number of the CYP2D6 gene, sub-genic region or pseudogene.

In certain aspects, copy number state for each given sample in the reference set is predicted as the maximal a posteriori copy number state. In certain aspects, a transfer learning approach is applied to adapt a learned Gaussian mixture model to a new set of samples. In certain aspects, the method comprises retaining the means and variances of mixture components, and updating the class priors in the Gaussian mixture model based on the new sample set. In certain aspects, the nucleotide sequence information comprises whole genome sequencing (WGS) data. In certain aspects, the nucleotide sequence information comprises microarray data.

In certain aspects, the microarray data is obtained using one or more microarrays selected from: Infinium Global Screening Array v2.0 (GSAv2) and All of Us (AoU) Infinium Global Diversity Array. In certain aspects, the microarray data is obtained using a microarray comprising at least 1.8M SNPs. In certain aspects, the microarray data is obtained using a microarray comprising multi-ethnic SNPs. In certain aspects, the subject is a fetal subject, a neonatal subject, a pediatric subject, or an adult subject. In certain aspects, the sample comprises cells or cell-free DNA.

In certain aspects, a sequence read of the plurality of sequence reads is aligned to the CYP2D6 gene or the CYP2D7 gene with an alignment quality score of about zero. In certain aspects, the method comprises determining a treatment recommendation for the subject based on the copy number of the SMN1 gene determined. In certain aspects, the method comprises determining a dosage recommendation of a treatment and/or a treatment recommendation for the subject based on at least one of the small variant and the structural variant.

Also presented herein is a method for copy number estimation of a target gene with close homologs, comprising determining sub-genic copy numbers of said target gene and/or said close homologs. In certain aspects, the target gene is a functional gene. In certain aspects, one or more of the homologs comprises a non-functional pseudogene. In certain aspects, one or more of the homologs comprises pseudogene with structural variations.

In certain aspects of the above embodiments, the method comprises, under control of a hardware processor: receiving quantitative data comprising nucleotide sequence information at one or more specific sites of the target gene, said quantitative data obtained from a sample of a subject analyzed; determining a first number of informative signals from each of said one or more specific sites; determining a first normalized number of informative signals from each of said one or more specific sites; determining an aggregated informative signal for each of a plurality of target regions, and determining a total copy number of one or more target genes, sub-genic regions or pseudogenes using a Gaussian mixture model.

Also presented herein is a computer system for copy number estimation of a target gene with close homologs, the system comprising computer-readable instructions for determining sub-genic copy numbers of said target gene and/or said close homologs.

In certain aspects, the computer-readable instructions comprise instructions for: receiving quantitative data comprising nucleotide sequence information at one or more specific sites of the target gene, said quantitative data obtained from a sample of a subject analyzed; determining a first number of informative signals from each of said one or more specific sites; determining a first normalized number of informative signals from each of said one or more specific sites; determining an aggregated informative signal for each of a plurality of target regions, and determining a total copy number of one or more target genes, sub-genic regions or pseudogenes using a Gaussian mixture model.

The details of one or more embodiments are set forth in the accompanying drawings and the description below. Other features, objects, and advantages will be apparent from the description and drawings, and from the claims.

### BRIEF DESCRIPTION OF THE DRAWINGS

FIG. 1 is a block diagram of an illustrative computing system configured to implement diagnosing from array data or whole genome sequencing data.

FIG. 2 shows CNV calling accuracy for CYP2D6 using prior art tools.

FIG. 3 shows an architecture of one example CNV calling system configured according to one implementation of the methods presented herein.

FIG. 4A is a schematic showing various CYP2D6/7 fusion genes and FIG. 4B is a table showing a probe design strategy for microarray detection of each region.

### DETAILED DESCRIPTION

In this disclosure, methods for accurate sub-genic CNV calling with a set of reference samples are described. The example below illustrates one implementation of the claimed methods. Specifically, methods for accurate sub-genic CYP2D6 CNV calling with a set of reference samples are described. It will be appreciated by those of ordinary skill in the art that the methods can be generalized to other genes of similar, greater, or less complexity. Existing tools for CNV calling have difficulty calling these regions due to common gene conversions between CYP2D6 and CYP2D7 (referred to as CYP2D6/7 hereafter), common SVs (gene deletions, duplications and CYP2D6/7 fusion genes), as well as the sequence similarity between CYP2D/7, which results in ambiguous read alignments to either genes. Some existing callers cannot detect complex structural variants and have been shown to have low performance.

### Execution Environment

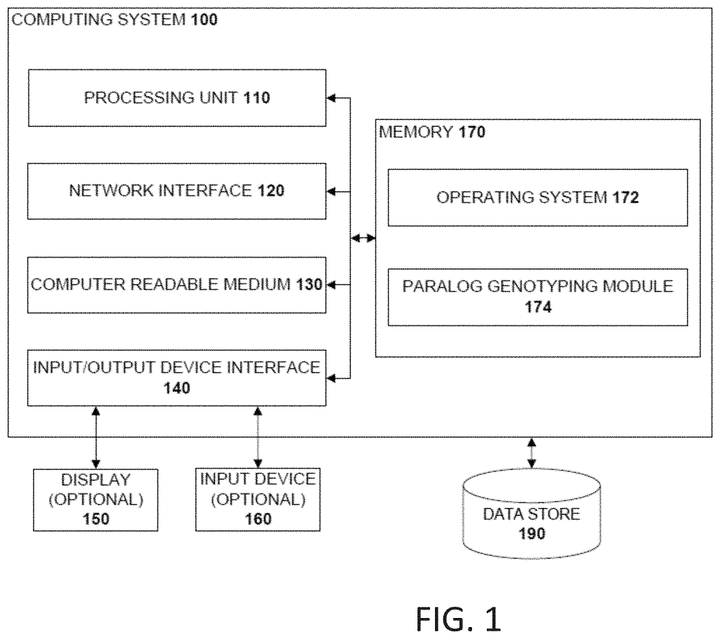

FIG. 1 depicts a general architecture of an example computing device 100 configured to implement the CNV calling system disclosed herein. The general architecture of the computing device 100 depicted in FIG. 1 includes an arrangement of computer hardware and software components. The computing device 100 may include many more (or fewer) elements than those shown in FIG. 1. It is not necessary, however, that all of these generally conventional elements be shown in order to provide an enabling disclosure. As illustrated, the computing device 100 includes a processing unit 110, a network interface 120, a computer readable medium drive 130, an input/output device interface 140, a display 150, and an input device 160, all of which may communicate with one another by way of a communication bus. The network interface 120 may provide connectivity to one or more networks or computing systems. The processing unit 110 may thus receive information and instructions from other computing systems or services via a network. The processing unit 110 may also communicate to and from memory 170 and further provide output information for an optional display 150 via the input/output device interface 140. The input/output device interface 140 may also accept input from the optional input device 160, such as a keyboard, mouse, digital pen, microphone, touch screen, gesture recognition system, voice recognition system, gamepad, accelerometer, gyroscope, or other input device.

The memory 170 may contain computer program instructions (grouped as modules or components in some embodiments) that the processing unit 110 executes in order to implement one or more embodiments. The memory 170 generally includes RAM, ROM and/or other persistent, auxiliary or non-transitory computer-readable media. The memory 170 may store an operating system 172 that provides computer program instructions for use by the processing unit 110 in the general administration and operation of the computing device 100. The memory 170 may further include computer program instructions and other information for implementing aspects of the present disclosure.

For example, in one embodiment, the memory 170 includes a genotyping module 174 for genotyping one or more homologs or paralogs, such as determining a copy number of survival of motor neuron 1 (SMN1) gene and/or genotyping cytochrome P450 Family 2 Subfamily D Member 6 (CYP2D6). In addition, memory 170 may include or communicate with the data store 190 and/or one or more other data stores that sequencing data.

### EXAMPLE 1

### Methods for Accurate Sub-Genic CYP2D6 CNV Calling with a Set of Reference Samples

This example describes one implementation of the claimed methods. For many clinically important genes, copy number estimation can be challenging due to high sequence similarity between gene of interest and their homologs, including non-functional pseudogenes.

There is significant variation in the response of individuals to a large number of clinically prescribed drugs. A strong contributing factor to this differential drug response is the genetic composition of the drug-metabolizing genes. Precision medicine requires genotyping pharmacogenes to enable personalized treatment. Cytochrome P450 2D6 (CYP2D6) is one of the most important drug-metabolizing genes and is involved in the metabolism of 25% of drugs. The CYP2D6 gene is highly polymorphic, with 106 star alleles defined by the Pharmacogene Variation (PharmVar) Consortium (pharmvar.org/gene/CYP2D6). CYP2D6 star alleles are CYP2D6 gene copies defined by a combination of small variants (such as single nucleotide variations (SNVs) and insertions/deletions (indels)) and structural variants (SVs), and correspond to different levels of CYP2D6 enzymatic activity, such as poor, intermediate, normal, or ultrarapid metabolizer.

For example, CYP2D6 copy number determination is essential for determining the drug metabolizer status of CYP2D6. It is required for the implementation of pharmacogenomics or precision medicine. However, accurate CYP2D6 CNV calling is challenging due to the presence of two nearby pseudogenes and cooccurrence of multiple types of structural variations. In order to differentiate the copies of functional CYP2D6 alleles against the non-functional alleles with structural variations, we need sub-genic resolution in copy number estimation, e.g. copy numbers for specific introns and exons.

The genotyping of CYP2D6 is further challenged by the presence of a nonfunctional paralog, CYP2D7, that is located upstream of CYP2D6 and shares 94% sequence similarity, with a few near-identical regions. Traditionally, CYP2D6 genotyping has been done with arrays or polymerase chain reaction (PCR) based methods, such as TaqMan assays, droplet digital PCR (ddPCR) and long-range PCR. These assays often have difficulty detecting structural variants. The methods presented herein provide significant improvements in the ability to call CNV and estimate copy number, as desctibed below.

### Signal Aggregation

Whether it is with an array or next-generation sequencing (NGS), for a predefined target region associated a given gene, the intensity signal from an array (or counts of sequence reads) from all nucleotides falling into this region are collected, and then only the signal coming from target gene specific nucleotide is used. Such signals are referred to as informative signals. For example, if a probe is designed to produce signals from both target gene and off-target genes, only the signal specific to the target gene is the informative signal. Standard signal normalization from array or NGS are applied and genomic GC content-based normalization is applied before extracting the informative signals.

For each target region, the extracted informative signals are aggregated through the arithmetic mean:

Tₛ=Σₛₗ Rₛₗ/L, or Tₛ=Σₛₗ exp(rₛₗ)/L

where r and R are the log scale or linear scaled normalized signal respectively, s and l indicate the sample and (informative) loci, and L is the total number of loci.
Alternatively, geometric mean can be used:

Tₛ=exp(Σₛₗ log(Rₛₗ)/L)

In some preferred embodiments, the arithmetic mean is used. In certain embodiments, the arithmetic mean can perform slightly better than geometric mean, and performance improves with increasing L.

Alternatively, a weighted version of the signal aggregation method is applied to achieve better outlier resistance,

Tₛ=ρₗ rₛₗ/σₗ²

Where σₗ² is the variance of signal of a given loci across all samples.

Following signal aggregation, a centering step was applied to remove batch effect common to all samples.

### Restricted Expectation Maximization (EM) Algorithm

An unsupervised machine learning method was used to model the aggregated signals to enable better copy number prediction for a target region. Given a reference set samples of size S, the aggregated signal Tₛ for s in 1 . . . S was used with a Gaussian mixture model.

Given that the intensity signal differences between different copy number status are small compared to the variations of the intensity signals, a standard expectation maximization (EM) algorithm for Gaussian mixture model does not yield stable results. Therefore, a restricted EM algorithm was developed to enforce expected (mean) signal intensity to be within prespecified range for each copy number state. Briefly, the restricted EM algorithm is as following:
1. Multiple iterations of EM-restriction are performed;
2. In each EM-restriction iteration,
   a. standard EM algorithm is performed until convergence criteria is met.
   b. after that, EM-restriction is applied such that
      i. For multiple mixture components that fall into the same range (see Table 1 for example set of ranges), these components are merged into one component
      ii. For a given range that has no component, a new component is created based on the initial values (Table 1).
3. EM-Restriction interactions are repeated until convergence.

Parameters for restricted EM algorithm for log scale intensity.

| | Lower bound | Initial Value | Upper bound |
|---|---|---|---|
| 0 | −10 | −1.3 | −1.0 |
| 1 | −1 | −0.4 | −0.1 |
| 2 | −0.1 | 0 | 0.1 |
| 3 | 0.1 | 0.2 | 0.3 |
| 4 | 0.3 | 0.4 | 0.5 |
| 5 | 0.5 | 0.7 | 10 |

Note that the restricted EM method will estimate the means and variances of intensity signals associated with difference copy number states, and it also estimates the priors associated to the copy number states.

### Transfer Learning Method for Copy Number Prediction

After we construct the Gaussian mixture model (GMM) for aggregated EM algorithm using the restricted EM algorithm, copy number state for each given sample in the reference set is predicted as the maximal a posteriori copy number state.

To make prediction on a new set of samples, a transfer learning approach is applied to adapt the learned GMM to the new set. Specifically, we retain the means and variances of mixture components, and update the class priors in the GMM based on the new sample set.

### Results

The results of the above-described methodology are set forth in Table 2 below. Two different bead chip arrays were used: Infinium Global Screening Array v2.0 (GSAv2) and All of Us (AoU) Infinium Global Diversity Array, (Illumina, San Diego, Calif.). The AoU array is a 1.8M SNP array that includes a diverse set of multi-ethnic SNPs, including 88,263 ClinVar and/or ACMG 59 SNPs (including 28,428 ClinVar Pathogenic SNPs), 14,980 Disease & Predisposition (NHGRI) SNPs, 18,730 HLA/KIR SNPs, 29,571 PGx (ADME-CPIC, PharmGKB) SNPs, and a set of 1,332,680 Genome Wide Backbone SNPs. For comparison, the GSA array is a 0.7M SNP array that includes 55,385 ClinVar and/or ACMG 59 variants, 10,574 Disease & Predisposition (NHGRI) SNPs, 8,577 HLA/KIR SNPs, 17,220 PGx (ADME-CPIC, PharmGKB) SNPs, and a set of 544K Genome Wide Backbone SNPs.

As demonstrated in Table 2, CNV calling accuracy for intron 2 of CYP2D6 ranged from 65% to 100%, depending on the bead chip and sample set. Similarly, CNV calling accuracy for intron 6 of CYP2D6 ranged from 88.9% to 98.2%, and accuracy for exon 9 ranged from 80% to 100%, depending on the bead chip and sample set. Overall CNV calling accuracy for CYP2D6 ranged from 84.5% to 99.5%, depending on the bead chip and sample set. Copy Number Truth for these cell lines were determined by orthogonal technologies, including TaqMan assay and PacBio SMRT seq.

TABLE 2
Summary of CNV calling accuracy based on two sets of samples
on two different BeadChips. Set. 1 or Set. 2 corresponds
to 41 cell lines (58 samples) and 115 cell lines (115 samples)
respectively. CNV calling accuracy is measured by F-measure
of corrected predicted copy number gains or losses.

| | Cell | | Intron | Intron | Exon | All |
|---|---|---|---|---|---|---|
| Chips | Lines | CYP2D6 | 2 | 6 | 9 | combined |
| GSAv2 | 41 | 89.8% | 65.0% | 98.1% | 80.0% | 84.5% |
| Set. 1 | | | | | | |
| AoU Set. 1 | 41 | 100% | 100% | 98.2% | 100% | 99.5% |
| AoU Set. 2 | 115 | 86.6% | 83.3% | 88.9% | 82.8% | 85.5% |

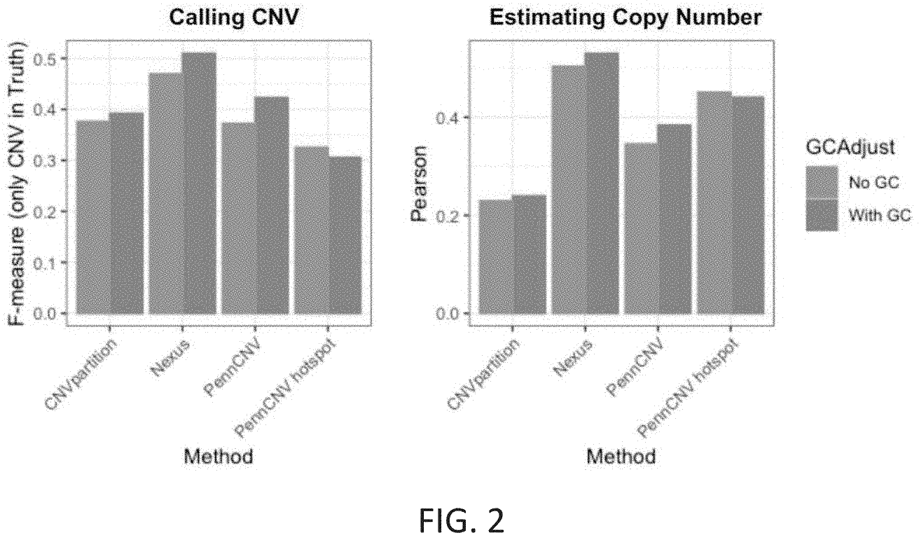

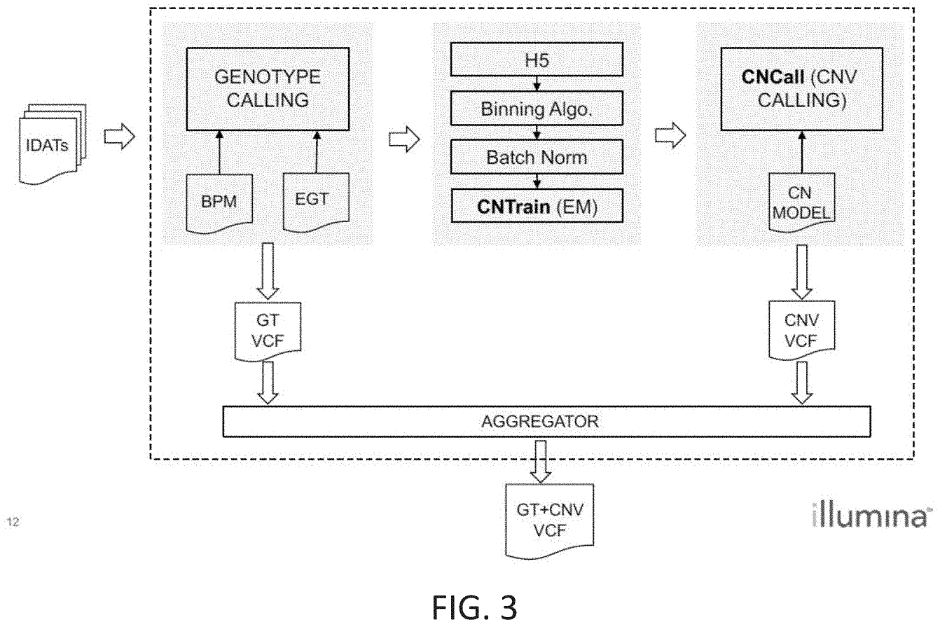

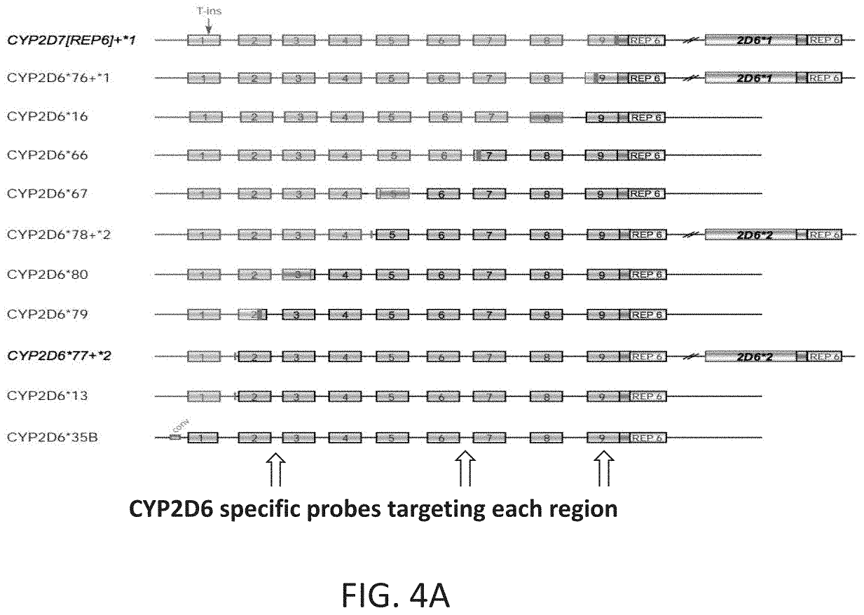

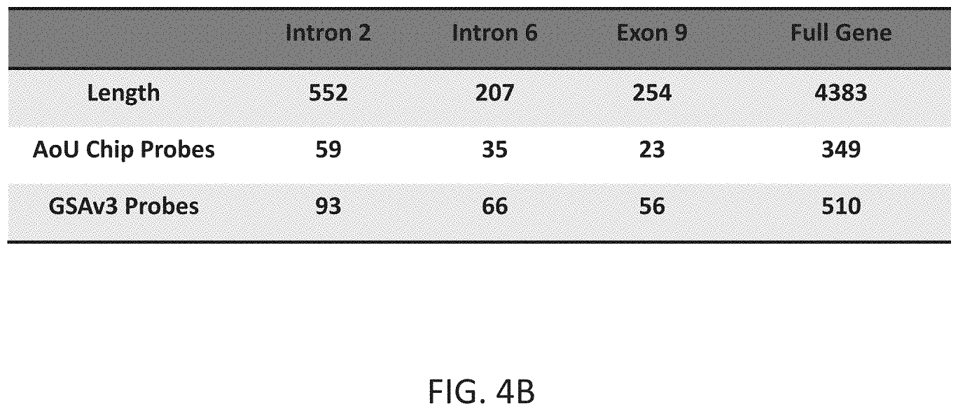

These data confirm that the approach set forth herein can be successfully used to differentiate the copies of functional CYP2D6 alleles against the non-functional alleles with structural variations. By obtaining sub-genic resolution in copy number estimation for specific introns and exons, a high degree of CNV calling accuracy is obtained. Prior art tools such as CNVpartition, Nexus, PennCNV, and PennCNV hotspot have poor overall CNV calling accuracy for CYP2D6, with F-measure scores ranging from around 30% to around 50% (FIG. 2). In contrast the method presented herein is able to obtain F-measure scores greater than 84% and even as high as 99.5%, as shown in Table 2. These scores represent a dramatic improvement in CNV calling accuracy.

Throughout this application various publications, patents and/or patent applications have been referenced. The disclosure of these publications in their entireties is hereby incorporated by reference in this application.

The term comprising is intended herein to be open-ended, including not only the recited elements, but further encompassing any additional elements.

A number of embodiments have been described. Nevertheless, it will be understood that various modifications may be made. Accordingly, other embodiments are within the scope of the following claims.

What is claimed is:

1. A method for genotyping cytochrome P450 Family 2 Subfamily D Member 6 (CYP2D6) gene comprising:
under control of a hardware processor:
receiving quantitative data comprising nucleotide sequence information at one or more specific sites of cytochrome P450 Family 2 Subfamily D Member 6 (CYP2D6) gene or cytochrome P450 Family 2 Subfamily D Member 7 (CYP2D7) gene, said quantitative data obtained from a sample of a subject analyzed;
determining a first number of informative signals from each of said one or more specific sites;
determining a first normalized number of informative signals from each of said one or more specific sites
determining an aggregated informative signal for each of a plurality of target regions, and
determining a total copy number of one or more CYP2D6 genes, sub-genic regions or pseudogenes using a Gaussian mixture model.

2. The method of claim 1, wherein determining (i) a first normalized number of informative signals comprises normalizing based on the length of a gene or sub-genic region.

3. The method of claim 1, wherein determining (i) a first normalized number of informative signals comprises normalizing based on genomic GC content of a gene or sub-genic region.

4. The method of claim 1, wherein the extracted informative signals are aggregated through an arithmetic mean.

5. The method of claim 4, wherein the arithmetic mean comprises:
Tₛ=Σₛₗ Rₛₗ/L, or Tₛ=Σₛₗ exp(rₛₗ)/L
where r and R are the log scale or linear scaled normalized signal respectively, s and l indicate the sample and (informative) loci, and L is the total number of loci.

6. The method of claim 1, wherein the extracted informative signals are aggregated through a geometric mean.

7. The method of claim 6, wherein the geometric mean comprises:
Ts=exp(Σsl log(Rsl)/L).

8. The method of claim 1, wherein a weighted version of the signal aggregation method is applied.

9. The method of claim 8, wherein the weighted version of the signal aggregation method comprises:
Ts=Σl rsl/σl2
where σl2 is the variance of signal of a given loci across all samples.

10. The method of claim 1, further comprising, following signal aggregation, a centering step to remove batch effect common to all samples.

11. The method of claim 1, wherein the Gaussian mixture model comprises a restricted expectation maximization (EM) algorithm.

12. The method of claim 11, wherein the restricted EM algorithm estimates the means and variances of intensity signals associated with difference copy number states.

13. The method of claim 11, wherein the restricted EM algorithm estimates the priors associated to the copy number states.

14. The method of claim 1, wherein the Gaussian mixture model comprises a plurality of Gaussians each representing a different integer copy number, given the first normalized number of the quantitative sequence information from the one or more specific sites of the CYP2D6 gene.

15. The method of claim 1, wherein determining a total copy number of one or more CYP2D6 genes, sub-genic regions or pseudogenes comprises, for one of a plurality of CYP2D6 gene-specific bases, determining a most likely combination, of a plurality of possible combinations each comprising a possible copy number of the CYP2D6 gene, sub-genic region or pseudogene.

16. The method of claim 1, wherein copy number state for each given sample in the reference set is predicted as the maximal a posteriori copy number state.

17. The method of claim 1, wherein a transfer learning approach is applied to adapt a learned Gaussian mixture model to a new set of samples.

18. The method of claim 17, comprising retaining the means and variances of mixture components, and updating the class priors in the Gaussian mixture model based on the new sample set.

19. The method of claim 1, wherein the nucleotide sequence information comprises whole genome sequencing (WGS) data.

20. The method of claim 1, wherein the nucleotide sequence information comprises microarray data.

21. The method of claim 20, wherein the microarray data is obtained using one or more microarrays selected from: Infinium Global Screening Array v2.0 (GSAv2) and All of Us (AoU) Infinium Global Diversity Array.

22. The method of claim 20, wherein the microarray data is obtained using a microarray comprising at least 1.8M SNPs.

23. The method of claim 20, wherein the microarray data is obtained using a microarray comprising multi-ethnic SNPs.

24. The method of claim 1, wherein the subject is a fetal subject, a neonatal subject, a pediatric subject, or an adult subject.

25. The method of claim 1, wherein the sample comprises cells or cell-free DNA.

26. The method of claim 1, wherein a sequence read of the plurality of sequence reads is aligned to the CYP2D6 gene or the CYP2D7 gene with an alignment quality score of about zero.

27. The method of claim 1, comprising determining a treatment recommendation for the subject based on the copy number of the SMN1 gene determined.

28. The method of claim 1, comprising determining a dosage recommendation of a treatment and/or a treatment recommendation for the subject based on at least one of the small variant and the structural variant.

29. A method for copy number estimation of a target gene with close homologs, comprising determining sub-genic copy numbers of said target gene and/or said close homologs.

30. The method of claim 29, wherein the target gene is a functional gene.

31. The method of claim 29, wherein one or more of the homologs comprises a non-functional pseudogene.

32. The method of claim 29, wherein one or more of the homologs comprises pseudogene with structural variations.

33. The method of claim 29, comprising, under control of a hardware processor:
receiving quantitative data comprising nucleotide sequence information at one or more specific sites of the target gene, said quantitative data obtained from a sample of a subject analyzed;
determining a first number of informative signals from each of said one or more specific sites;
determining a first normalized number of informative signals from each of said one or more specific sites
determining an aggregated informative signal for each of a plurality of target regions, and
determining a total copy number of one or more target genes, sub-genic regions or pseudogenes using a Gaussian mixture model.

34. A computer system for copy number estimation of a target gene with close homologs, the system comprising computer-readable instructions for determining sub-genic copy numbers of said target gene and/or said close homologs.

35. The system of claim 34, wherein the computer-readable instructions comprise instructions for:
receiving quantitative data comprising nucleotide sequence information at one or more specific sites of the target gene, said quantitative data obtained from a sample of a subject analyzed;
determining a first number of informative signals from each of said one or more specific sites;
determining a first normalized number of informative signals from each of said one or more specific sites
determining an aggregated informative signal for each of a plurality of target regions, and
determining a total copy number of one or more target genes, sub-genic regions or pseudogenes using a Gaussian mixture model.
